(19) United States
(12) Patent Application Publication
(10) Pub. No.: US 2015/0316568 A1
(43) Pub. Date: Nov. 5, 2015

(54) Method for Screening Lyophilized Parenteral Formulations

(71) Applicant: Cubist Pharmaceuticals, Inc., Lexington, MA (US)

(72) Inventors: Mingjin Fan, Lexington, MA (US); Ujjwal Joshi, Waltham, MA (US); Jonathan Cam Ly, Arlington, MA (US); Akash Jain, Burlington, MA (US)

(21) Appl. No.: 14/701,229

(22) Filed: Apr. 30, 2015

Related U.S. Application Data
(60) Provisional application No. 61/986,537, filed on Apr. 30, 2014.

(51) Int. Cl. G01N 33/94; A61K 31/4525; A61K 31/496

(52) U.S. Cl. CPC G01N 33/9446; A61K 31/496; A61K 31/4525; G01N 33/9466

(57) ABSTRACT

Embodiments of the present invention feature methods for screening parenteral formulations which require small quantities of drug and rapid results. The method features steps of identifying one or more solutions for reconstitution and applying the solutions to lyophilized drug, excipient and diluent combinations.

### RELATED APPLICATION

This application claims priority to U.S. Provisional Application No. 61/986,537, filed on Apr. 30, 2014. The entire content of this application is incorporated herein by reference in its entirety.

### FIELD OF INVENTION

Embodiments of the present invention relate to lyophilized compounds which are stored for a period of time and rehydrated for use. The compounds, sometimes referred herein as a compound of interest, are in the nature of a drug or pharmaceutical used to treat disease conditions or effect biological functions in humans and animals.

### BACKGROUND OF THE INVENTION

Biologically active compounds that are potential candidates for pharmaceutical products need to be formulated for delivery to the body. The formulation should preserve the activity and produce a consistent and cost effective response. Many drugs are administered parenterally due to instability, or lack of absorption through other routes of administration or as the most efficient manner of administering the drug. As used herein, the term “parenteral” refers to injection, for example, without limitation, by intravenous injection, intramuscular injection, intra-peritoneal injection, subcutaneous injection and the like.

Compounds that are administered parenterally are normally in solution. However, compounds in solution are more susceptible to degradation or may come out of solution over time. Many drug formulations for parenteral administration are held as lyophilized powders, for reconstitution at the time of administration or shortly before. Lyophilized forms of the compound of interest may be stable for prolonged periods of time. The term “lyophilized” refers to freeze drying type processes in which a material is frozen at cold temperatures and the pressure reduced to sublimate water and other liquids potentially present.

It is desirable to have parenteral drug formulations which are stable in the form stored and, if reconstituted from a lyophilized form, have the drug move to solution quickly and reproducibly, have the drug available in high concentration, and have such drug stable in the solution formed. These goals are difficult to achieve as a drug is being formulated into a new product for clinical studies and potential commercial applications.

Formulation development is complicated by batch to batch variability of the drug composition. And, the drug composition may be available in limited quantities.

It would be desirable to have methods and processes for rapidly and efficiently identifying one or more potential formulations in which the compound of interest is lyophilized for reconstitution at or shortly before parenteral administration.

### SUMMARY OF THE INVENTION

Embodiments of the present invention are directed to methods for identifying one or more solutions for rehydration, excipients, and diluents for use with a compound of interest in a lyophilization process and rehydration process. That is, the excipients and diluents used with the compound of interest as part of a parenteral formulation, and the solution for rehydration are readily identified in a rapid, efficient high throughput screening process. One embodiment of the method comprises the steps of providing a plurality of wells for performing lyophilization processes. The plurality of wells is divided into well groups, corresponding to at least a first well group and at least one second well group. The first well group has at least one well for receiving each test rehydrating solution. The second well group has at least one well for receiving a quantity of a test excipient and/or diluent. An aliquot of a compound of interest is placed in each well of the first well group and of the second well group. The aliquot has a concentration of the compound of interest at or above the desired concentration of a parenteral formulation. Each well of the second well group receives a quantity of a test excipient and/or diluent. And, the compound of interest and the test excipients and/or diluent are placed in solution. Next, lyophilization conditions are imposed on the wells to produce a plurality of lyophilized samples comprising lyophilized compound of interest in wells of the first well group, and lyophilized compound of interest with a test excipient and/or diluent in the wells of the second well group. Next, each well of the first well group is rehydrated with a test rehydrating solution and at least one preferred rehydrating solution is identified. Next, each well of the second well group is rehydrated with the at least one preferred rehydrating solution to identify at least one preferred excipient and/or diluent for use with the at least one identified preferred rehydrating solution for use in a parenteral formulation of the compound of interest.

As used herein, the term “lyophilization conditions” means placing the well in an environment in which the contents of the well will undergo lyophilization. Lyophilization processes and conditions with respect to vacuum and temperature are well known in the art. The term “placed” is used to means put into, such as made into a solution or put into a well.

A preferred rehydrating solution and/or a preferred excipient and/or diluent is normally one that facilitates the compound of interest quickly entering solution, staying in solution for a period of time allowing for the administration of the drug, producing a clear solution, and solubilizing a larger quantity of drug to produce a concentrated solution. Excipients and diluents are inactive ingredients which facilitate the compound of interest entering solution. The present discussion does not distinguish between excipients and diluents and the terms are synonymous for all purposes herein.

Steps and features of the present method are readily automated or are made semi-automated. For example, without limitation, one embodiment of the present method features the plurality of wells constructed and arranged in a 96-well format and multiples of 96-wells. Lyophilization conditions are imposed by an automated process in which the wells, divided into at least a first well group and at least one second well group, are subjected to substantially the same conditions. For example, a row or a line of the 96-well arrangement is designated a first well group and the remainder rows and/or lines are designated the second well group.

One embodiment of the present method features the step of identifying preferred lyophilization process conditions by forming two or more clusters each cluster comprising a first well group and a second well group. That is, one 96-well device may comprise a first cluster and a second 96-well device may comprise a second cluster. Each cluster is subjected to a different lyophilization process. For example without limitation, the period of vacuum, the degree of vacuum, the temperature, the time period of the temperature, or any combination thereof may be different.

The clusters can also be used to identify an excipient and/or diluent or rehydration solution exhibiting a desired stability. That is, each cluster can be held for a period of time prior to, or after, rehydration to evaluate the effect of such period.

A further embodiment of the present invention is directed to a formulation of a compound of interest identified by the present processes or a rehydration solution used with a formulation of a compound of interest identified by the present process or a lyophilization process identified by the present processes.

A further embodiment of the present invention features a method of increasing the solubility of a pharmaceutical agent. The method comprises the steps of dissolving a pharmaceutical agent in a first solute, in which the pharmaceutical agent exhibits a first solubility in such first solute; and lyophilizing the pharmaceutical agent in the first solute to form a lyophilized composition comprising the pharmaceutical agent. The lyophilized composition has a second solubility in the first solute that is greater than the first solubility.

One embodiment of the present method features water as the first solute, and the lyophilized composition is obtained by lyophilizing an aqueous solution of the pharmaceutical agent. The examples feature the pharmaceutical agents: ciprofloxacin, paroxetine, and difloxacin. The method further comprises adding one or more excipients to the first solute prior to lyophilization.

Thus, the present inventions are directed to methods and processes for rapidly and efficiently identifying one or more potential formulations in which the compound of interest is lyophilized, for reconstitution at or shortly before parenteral administration. These and other features and advantages will be apparent to those skilled in the art upon viewing the drawings briefly described in the text below and upon reading the detailed description of the invention that follows.

### BRIEF DESCRIPTION OF THE DRAWINGS

FIG. 1 is a flow diagram embodying features of the present invention;

FIG. 2 is a 96-well plate for use in the present method embodying features of the present invention;

FIG. 3 depicts a plate map having features of the present invention;

FIG. 4 depicts a spreadsheet displaying results in accordance with the present invention;

FIG. 5 depicts in graph form, the solubility improvement of Fleroxacin through features of the present invention;

FIG. 6 depicts a graph showing improvements in solubility of Fleroxacin with different excipients and diluents;

FIG. 7 depicts x-ray diffraction results pre-lyophilization for a select compound of interest;

FIG. 8 depicts x-ray diffraction results post lyophilization for a select compound of interest;

FIG. 9 depicts initial, post lyophilization and post reconstitution solubility results for Ciprofloxacin;

FIG. 10 depicts initial, post lyophilization and post reconstitution solubility results for Paroxetine;

FIG. 11 depicts initial, post lyophilization and post reconstitution solubility results for Difloxacin;

FIG. 12 depicts results of a test compound in various excipients and diluents;

FIG. 13 depicts x-ray diffraction results pre-lyophilization and post lyophilization (with an excipient) for a select compound of interest;

FIG. 14 depicts x-ray diffraction results pre-lyophilization and post lyophilization (with an excipient) for a select compound of interest;

FIG. 15 depicts a flow diagram of lyophilization cycle development embodying features of the present invention;

FIG. 16 depicts the chemical structure and certain chemical properties of fleroxacin;

FIG. 17 depicts reconstitution of a select compound of interest in a formulation embodying features of the present invention; and

FIG. 18 depicts pre-lyophilization and post lyophilization solubilities of a select compound of interest in a formulation embodying features of the present invention.

### DETAILED DESCRIPTION OF THE INVENTION

Embodiments of the present invention will now be discussed in detail with respect to a method for identifying one or more solutions for rehydration, excipients and diluents for use with a compound of interest in a lyophilization process and rehydration process. This discussion features what is now believed to be the preferred embodiments of the present invention and the best mode of the invention. However, these preferences may change over time and are subject to alteration and modification. Therefore the present disclosure should not be considered limiting but is merely exemplary of the present invention.

A lyophilization process has advantages including, but not limited to, ease of processing a liquid; enhanced product stability in a dry state; limited heating of the product for water removal; and rapid and easy dissolution of reconstituted product (solid form changed).

The present invention focuses on methods and processes directed to a high throughput lyophilization screening platform for enabling high dose parenteral formulations. Formulation development involves issues including, but not limited to, limited supply of material including discovery compounds; strict development timelines; batch to batch variations including morphic forms and crystallinity of material; problems using conventional approaches like pH adjustment, surfactants, co-solvents, and complexing agents for certain molecules; and potential unsuitability of methods like spray drying, rotary evaporation, nanomilling for thermolabile molecules. A high throughput lyophilization screening platform allows generation of stable amorphous forms of thermolabile active pharmaceutical ingredients in a 96-vial format; high kinetic solubility of amorphous forms upon reconstitution; enablement of the platform by a robust and relatively short lyophilization cycle; and the ability to screen multiple combinations of solubility and stability enhancing excipients including sugars, polymers, and amino acids.

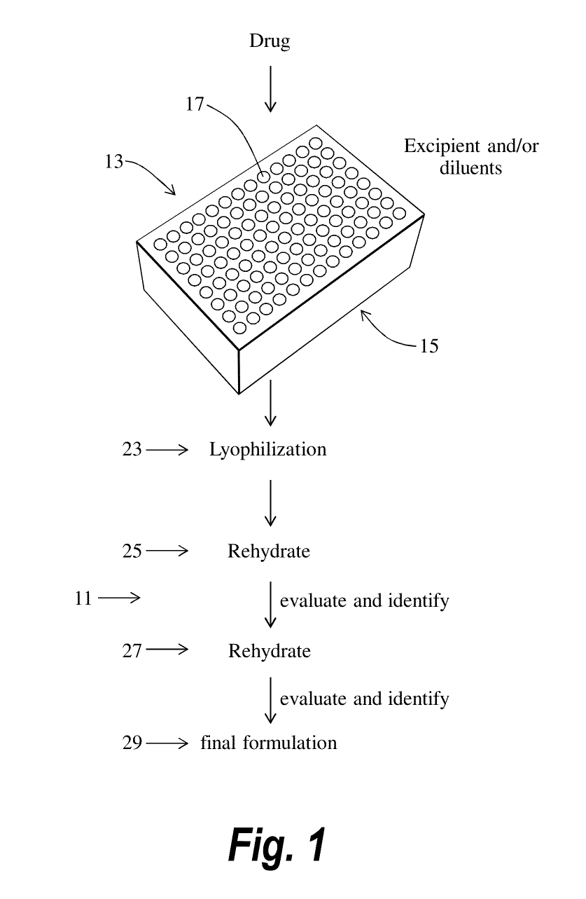

Turning now to FIG. 1, a flow diagram outlining features of the present method, designated by the numeral 11, is depicted. The method is used to identify the solution for rehydration, and excipients and diluents to be used with the compound of interest (sometimes referred herein as the drug) as part of a parenteral formulation.

In a first step 13, a plurality of wells for performing lyophilization processes is provided. As used herein, the term “provided” means acquired by or in possession of the user, as in available for his or her use. The plurality of wells may take many forms. In many drug formulation situations, the compound of interest is available in small quantities and is expensive to make. Embodiments of the present invention are well suited for relatively small, 2 mL vials, available from multiple vendors (for example, SP Industries Warminster, Pa. 18974), which require relatively small amounts of composition. Such vials can be arranged in a 96-well format with suitable racks, one such rack is designated by the numeral 15 in FIG. 1. For purposes of clarity only one horizontal row and one vertical line is shown in FIG. 1 having a plurality of vials 17. Those skilled in the art will recognize that other multiwell devices and formats can be readily substituted. Well formats in multiples of 96 are known in the art.

The plurality of wells is divided into well groups, corresponding to at least a first well group and at least one second well group. The 96-well format, and multiples of the 96-well format, can readily be divided by lines and rows or quadrants or blocks.

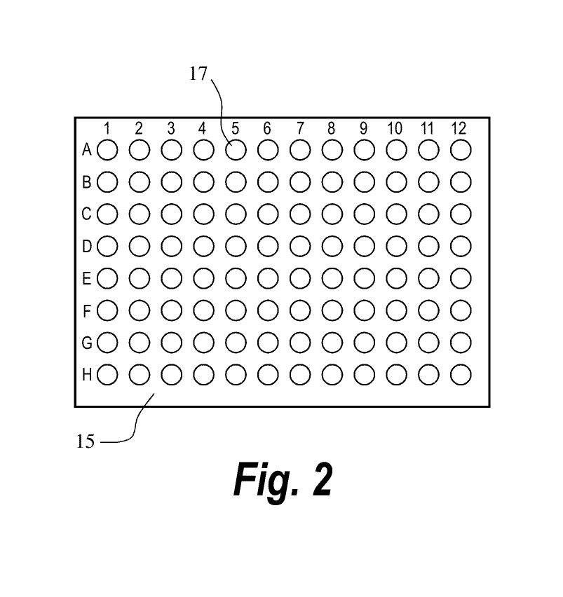

Turning now to FIG. 2, the rack 15 equipped with wells 17 is a 96-well device. This paper will term “rack” and “wells” and “multiwell device” interchangeably. The multiwell device 15 is depicted in top view having twelve lines of eight wells arranged vertically side by side numbered 1-12; and eight rows of twelve wells arranged horizontally numbered A-H.

For the purpose of this discussion, for example, without limitation, the first well group will be the first row “A” of the 96-well device 17. The first well group, row “A”, has at least one well for receiving each test rehydrating solution. If more rehydrating solutions than the number of vials are presented in row “A” are desired, one would add additional vials from other rows, or use multiple devices. For example, without limitation, the following rehydrating solutions are assigned to wells 1-12 of row “A”:

| Well | |
|---|---|
| Number | Rehydration solution |
| 1. | Sterile water for injection (sWFI) |
| 2. | 50 mM Acetate buffer, pH 5 |
| 3 | 5% TWEEN ® 80, q.s. sWFI |
| 4. | 10% TWEEN ® 80, q.s. sWFI |
| 5. | 5% CREMOPHOR ® EL, q.s. sWFI |
| 6. | 5% SOLUTOL ®, q.s. sWFI |
| 7. | 10% SOLUTOL ®, q.s. sWFI |
| 8. | 2% DMA, 5% SOLUTOL ®, q.s. sWFI |
| 9. | 5% DMA, 10% PEG 300, q.s. sWFI |
| 10. | 5% NMP, 10% PEG 300, q.s. sWFI |
| 11. | 5% NMP, 5% SOLUTOL ®, q.s. sWFI |
| 12. | 10% Ethanol, 10% CREMOPHOR ® EL, q.s. sWFI |

The second well group has at least one well for receiving a quantity of a test excipient and/or diluent. For example, the wells in rows B-H, are organized by excipient and/or diluent. For the purpose of this discussion, the three most promising rehydration solutions are preferred rehydration solutions for further evaluation. The wells for containing excipients and diluents are arranged in threes. In the event the number of preferred rehydration solutions for further evaluation is greater or less than three, the wells containing the excipients and diluents are arranged in accordance with such number. As used herein, the term arranged refers to being able to associate an excipient and/or diluent with a location. Thus, if one is working with a 96-well device and computer means, the computer may track the location although the actual wells may be far removed from each other. As used herein, “computer means” refers to computer processing units (CPUs) internal or external to a device, in the nature of servers, personal computers, hand held devices, tablets, laptop computers desktop computers and the like.

As depicted, row “B” will have three wells of Sucrose 5% w/v (lines 1-3), three wells of Sucrose 10% w/v (lines 4-6), three wells of Sucrose 15% w/v (lines 7-9) and three wells of Sucrose 20% w/v (lines 10-12). In a similar manner, row “C” presents escalating concentrations of Trehalose, row “D” presents an escalating concentration of polyvinylpyrrolidone (PVP) K, row “E” presents escalating concentrations of hydroxypropyl-β-cyclodextrin (HPCD), row “F” presents escalating concentrations of CAPTISOL® (modified cyclodextrin), row “G’ presents escalating concentrations of PLASDONE™ S-630, and row “H” presents escalating concentrations of Poloxamer 188.

An aliquot of a compound of interest is provided to each well of the first well group in row “A” and to the second well group rows “B-H”. The aliquot has an amount of the compound of interest to form a solution at or above the desired concentration of a parenteral formulation. For example, each vial would receive 2.2 mg of a compound of interest, and the vial would be filled to 100 μl for a final concentration of a drug formulation of 20 mg/mL. For a 96-well device with these desired concentrations, the amount of drug needed is 211.2 mg. This is a relatively small amount of drug to identify a potential parenteral formulation.

Each well of the second well group receives a quantity of a test excipient and/or diluent. The compound of interest and the test excipients and/or diluent are placed in solution. A multiwell device such as 96-well device 17 allows the imposition of solubilization steps and conditions in a substantially uniform manner across the device. For example, the vials may be maintained at substantially the same temperature and placed in shaking and sonification equipment.

Next, returning to FIG. 1, at step 23, lyophilization conditions are imposed on the wells to produce a plurality of lyophilized samples. The lyophilized samples comprise lyophilized compound of interest in wells of the first well group, row “A”. And, the lyophilized samples comprise lyophilized compound of interest with a test excipient and/or diluent in the wells of the second well group, rows “B-H”. Equipment for lyohilizing samples in a 96-well plate format are commercially available, for example, without limitation, VirTis® AdVantage™ Plus Personal Freeze Dryers (SP industries, USA).

If the stability of the lyophilized samples is to be evaluated, the multiwell device 17 is stored. If stability is not an issue, or is to be performed at a different time, the lyophilized samples of the first well group are rehydrated as indicated in the step designated by numeral 25. Each well of the first well group, row “A,” is rehydrated with a test rehydrating solution, as depicted in FIG. 1. Each rehydrated sample is evaluated. For example, without limitation, the amount of drug resolubilized is determined, the clarity of the resultant solution is noted, the ease of the lyophilized powder entering solution, and the presence of solids in the solution.

At least one preferred rehydrating solution is identified. In this discussion, the second well group was established for three preferred rehydrating solutions. Next, as shown in FIG. 1 as the step designated 27, each well of second well group is rehydrated with one of the three vials having the same excipient and/or diluent receiving one of the three preferred rehydrating solutions. For example, referring to FIG. 2, row “B’ line 1-3, are designated with the excipient Sucrose at 5% w/v. One vial, at row “B” line 1, receives one preferred rehydrating solution; one vial, at line 2, receives another rehydrating solution; and, one vial, at line 3, receives the last rehydrating solution. This is repeated for each excipient and/or diluent through the wells of the second well group.

The at least one preferred rehydrating solution is used to identify at least one preferred excipient and/or diluent for use with the identified at least one preferred rehydrating solution for use in a parenteral formulation of the compound of interest as shown as the step designated by numeral 29. A preferred rehydrating solution and/or a preferred excipient and/or diluent is normally one that facilitates the compound of interest quickly entering solution, staying in solution for a period of time allowing for the administration of the drug, producing a clear solution, and solubilizing a larger quantity of drug to produce a concentrated solution.

One embodiment of the present method features the step of identifying preferred lyophilization process conditions by forming two or more clusters each cluster comprising a first well group and a second well group. That is, more than one multiwell device 17 may be used. Each multiwell device comprises a cluster and each cluster is subjected to a different lyophilization process. For example without limitation, the period of vacuum, the degree of vacuum, the temperature, the time period of the temperature, or any combination thereof may be different.

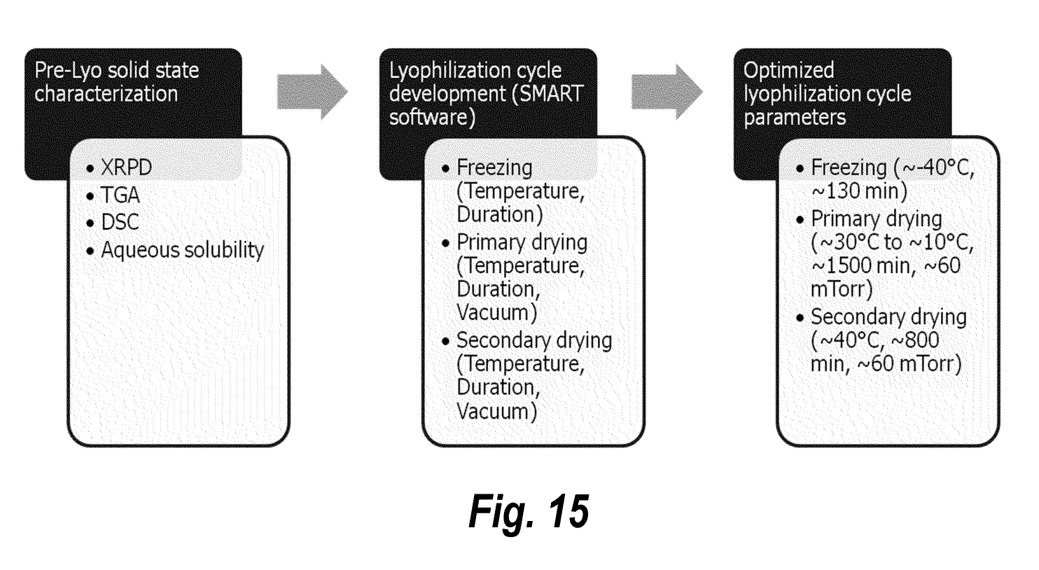

In FIG. 15, a flow diagram of lyophilization cycle development embodying features of the present invention is depicted. In some embodiments of the invention, the pre-lyophilization (pre-lyo) solid state of a select compound of interest may be characterized by one or more of the following: X-Ray Powder Diffraction (XRPD), thermogravimetric analysis (TGA), differential scanning calorimetry (DSC), and aqueous solubility. In certain embodiments, the lyophilization cycle may be developed to change one or more parameters of the freezing phase (e.g., temperature and duration), the primary drying phase (e.g., temperature, duration, and vacuum), and the secondary drying phase (e.g., temperature, duration, and vacuum), for example, using SMART software. In example embodiments, one or more parameters of the lyophilization cycle include a temperature of about −40 C. for about 130 min during the freezing phase, a temperature of about 30 C. to about 10 C. for about 1500 min at about 60 mTorr during the primary drying phase, and a temperature of about 40 C. for about 800 min at about 60 mTorr during the secondary drying phase.

Steps and features of the present method are readily automated or made semi-automated. For example, without limitation, one embodiment of the present method features the plurality of wells constructed and arranged in a 96-well format and multiples of 96-wells. Lyophilization conditions are imposed by an automated process in which the 96-wells, divided into at least a first well group and at least one second well group, are subjected to substantially the same conditions; or a first cluster, of a first well group and of a second well group, is subjected to a first set of lyophilization conditions and a second cluster, of a first well group and of a second well group, is subjected to a second set of lyophilization conditions.

A further embodiment of the present invention is directed to a formulation of a compound of interest identified by the present processes or a rehydration solution used with a formulation of a compound of interest identified by the present process or a lyophilization process identified by the present processes.

These and other features are highlighted in the Examples which follow.

### EXAMPLES

### Example 1

### Standard Procedures

The following methods and procedures were used in all examples unless expressly modified.

Aim: Use lyophilization as a high-throughput screening tool by making amorphous drug material.
Equipment: VirTis® AdVantage™ Plus (with a suitable filter unit for the organic solvents)
Containers: Glass vials (2.0 mL), the VirTis® Aluminum Microplate Frame, 96-well Lyophilization lids.
Stock solutions:

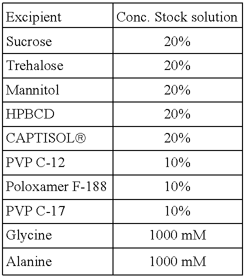

### Procedure:

### A) Setting up the Lyophilization:

1. Prepared all of the stock solution mentioned above.
2. Took the VirTis® Aluminum Microplate Frame and arranged 84 glass vials in the frame (leave the last lane empty).
3. Used the plate map and the Excel calculation sheet as reference to make all the calculations before starting the experiment, like:
a. Reconstitution volume
b. Concentration of the stock solution.
c. Amount of drug in each vial after lyophilization cycle.
d. Amount of respective excipient in each vial after lyophilization cycle.
e. % of each excipient after reconstitution.
4. Based on the calculations and on the plate map, added the concentrates of excipient solutions to respective vials. (Do not add any excipient solution to the first row).
5. Made a stock solution of the drug. For example, if the solubility of the drug in sterile water for injection (sWFI) was ˜5 mg/mL, a stock solution of 5 mg/mL was made.
6. Once the stock solution was ready, added the required amount of stock solution into each vial (also the first row).
7. For example, if your solution of the drug has a concentration of 5 mg/mL and 2.5 mg of the drug are desired in each vial after lyophilization, add 500 μL of the stock solution of the drug into each vial.
8. Placed the aluminum frame with all the vials on a shaker for 10 minutes to ensure proper mixing of all the solutions.
9. With a light hand, placed the 96-well lyophilization lids on the vials. Made sure they were sitting lightly on each and every vial without pressing hard.
10. Placed the frame into the Lyophilizer and started the lyophilization process using the following cycle:

### Exemplary Lyophilization Cycle

### Thermal Treatment

| Temperature (C.) | Time (min) | Ramp/hold |
|---|---|---|
| +05 | 20 | Ramp |
| +05 | 30 | Hold |
| −05 | 10 | Ramp |
| −05 | 30 | Hold |
| −40 | 35 | Ramp |
| −40 | 60 | Hold |

### Freeze, Condenser, Vacuum Phase

### Freeze=−40 C.

### Condenser=−45 C.

Vacuum=500 millitorr

### Drying Cycle Step:

| Temp(C.) | Time(Min) | Vacuum(millitorr) | Ramp/Hold |
|---|---|---|---|
| −40 | 120 | 60 | Hold |
| −32 | 15 | 60 | Ramp |
| −32 | 90 | 60 | Hold |
| −23 | 15 | 60 | Ramp |
| −23 | 135 | 60 | Hold |
| −10 | 30 | 60 | Ramp |
| −10 | 1200 | 60 | Hold |
| +40 | 495 | 60 | Ramp |
| +40 | 240 | 60 | Hold |

Storage: Temperature: +5 C., time 1200 minutes, vacuum 60 Millitorr
Total cycle time: 24 hours

### B) Reconstitution:

11. Once the lyophilization cycle was over, closed the lid system and removed the plate from the lyophilizer.
12. Placed the plate into a zip lock bag and stored at 4 C. until rehydration or reconstitution with a vehicle.

### Reconstitution Trial #1:

13. First, reconstituted only the first row of vials with the following reconstitution solutions and vortexed for 5-10 minutes:
a. sWFI
b. 50 mM Acetate buffer, pH˜5
c. 5% TWEEN®80, q.s. sWFI
d. 5% CREMOPHOR® EL, q.s. sWFI
e. 5% SOLUTOL®, q.s. sWFI
f. 2% DMA, 5% SOLUTOL®, q.s. sWFI
g. 2% DMA, 5% PEG 300, q.s. sWFI
h. 2% NMP, 5% PEG 300, q.s. WFI
i. 2% NMP, 5% SOLUTOL® in sWFI
j. 10% Ethanol, 10% CREMOPHOR® EL, q.s. sWFI
14. Made a note of physical appearance of each vial.
15. Filtered the solution and analyzed the solutions using HPLC.

| | Physical | Conc. |
|---|---|---|
| Sample | Observation | (mg/mL) |
| sWFI | | |
| 50 mM Acetate buffer, pH~5 | | |
| 10% TWEEN ®80 in sWFI | | |
| 10% CREMOPHOR ® in sWFI | | |
| 10% SOLUTOL ® in sWFI | | |
| 2% DMA, 5% SOLUTOL ®, | | |
| q.s. sWFI | | |
| 2% DMA, 5% PEG 300, q.s. sWFI | | |
| 2% NMP, 5% PEG 300, q.s. WFI | | |
| 2% NMP, 5% SOLUTOL ®, | | |
| q.s. sWFI | | |
| 10% Ethanol, 10% CREMOPHOR ® | | |
| EL, q.s. sWFI | | |

### Reconstitution Trial #2:

16. Based on the physical observations and the concentration of drug in a sample obtained, a reconstitution vehicle was chosen for the remaining vials. If all/or most of the vehicles gave similar concentration values, then the one with least amount of excipients was chosen.
17. Reconstituted the remaining vials, made a note of physical appearance and submitted for T0 concentration analysis.

| Sample | Physical Observation | Conc. (mg/mL) |
|---|---|---|
| Sucrose | | |
| Trehalose | | |
| Mannitol | | |
| HPBCD | | |
| CAPTISOL ® | | |
| PVP C-17 | | |
| PVP C-12 | | |
| Pluronic F-68 | | |
| Glycine | | |
| Alanine | | |

### Example 2

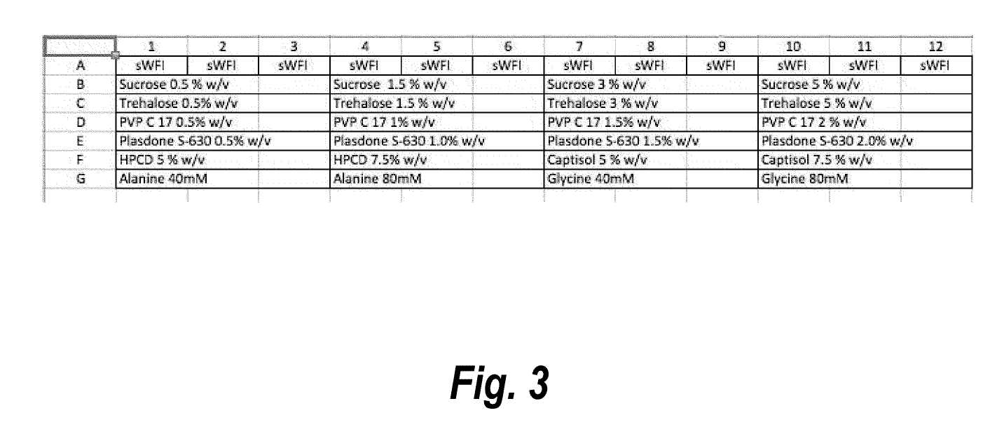

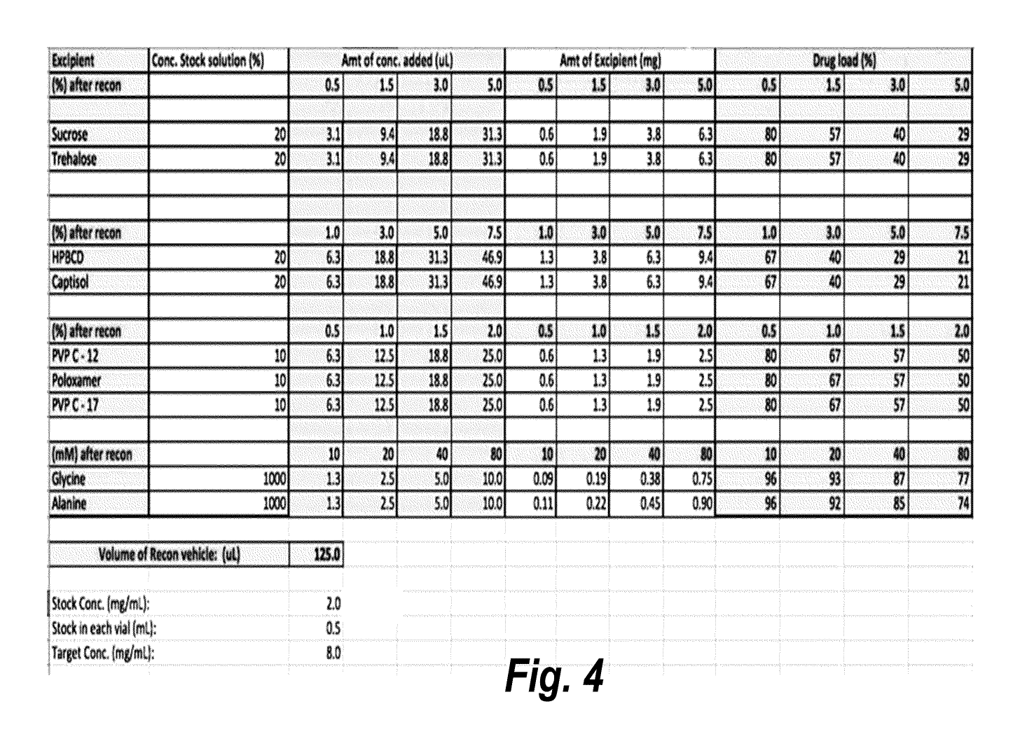

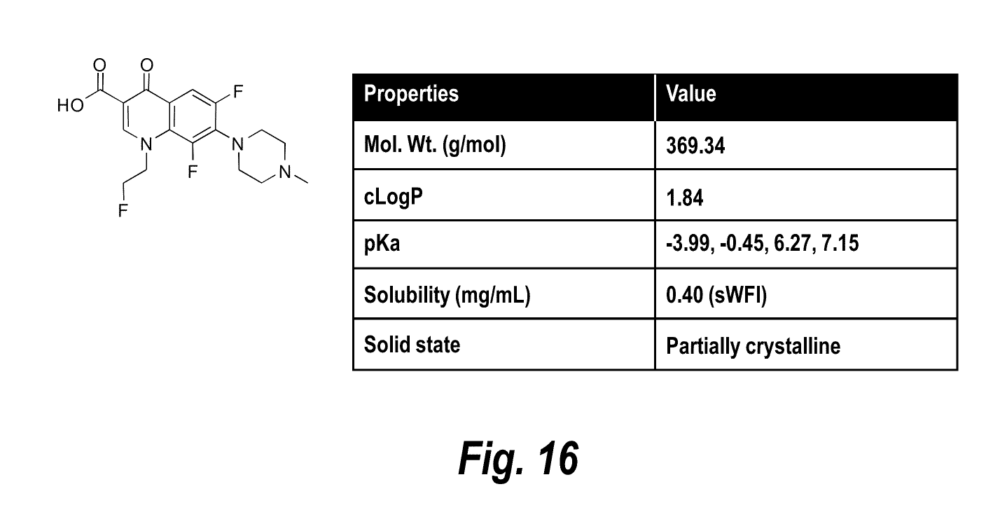

This example features the High Throughput Lyophilization of Fleroxacin and Paroxetine. Fleroxacin and Paroxetine are known compounds. FIG. 16 depicts the chemical structure and certain chemical properties of Fleroxacin.
After weighing out, 150.07 mg of Fleroxacin powder was slowly added to a stirring solution of 50-mLs of sWFI to aid dissolving.
Once the powder was added, the solution was stirred for 15 minutes to ensure that the crystals dissolved.
After dissolving, the solution was then vacuum filtered; the solid waste was removed.
Post-vacuum filtration, approximately 500 μL of the 5 mg/mL Paroxetine/sWFI solution was pipetted into 84 different lyophilization vials.
According to the plate map as depicted in FIGS. 3 and 4, and referring specifically to the columns for the amount of concentrate added (μL) of FIG. 4, the solutions were combined with selected excipients at various concentrations; the vials were made according to the tables.
The vials were vortexed until clear before going into the lyophilization machine (24 hours) loosely rubber capped.

### Post Lyophilization Reconstitution

After 24 hours, the vials were removed from the lyophilization machine for reconstitution.
All lyophilization vials were mixed with the assigned 100.0 μL of sWFI
Upon contact with sWFI, the solidified and frozen crystals immediately collapsed into solution.
The entire volume of sample from the lyophilization vial was transferred to a centrifuge tube.
After 2 minutes of centrifugation, 50 μL of supernatant was added to small HPLC vials and further diluted with an additional 450 μL sWFI.
All vials, labeled A1-G12, were capped and vortexed.
After reconstitution, the vials were placed in HPLC for analysis.

### Compound: Fleroxacin

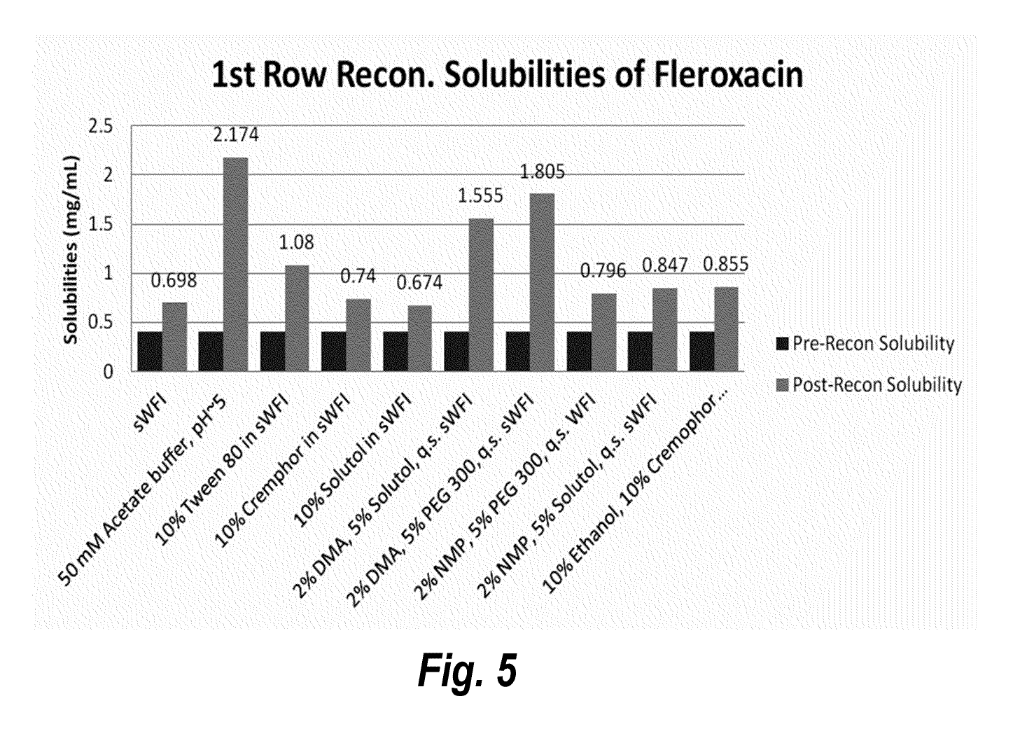

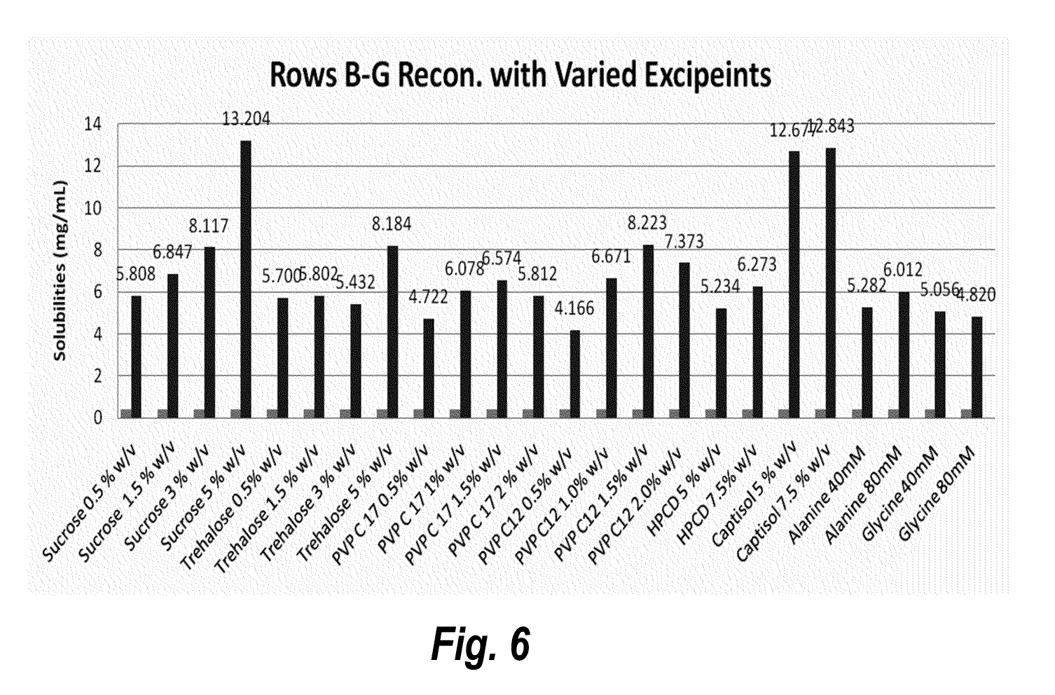

As shown in the Graph of FIG. 5, the 50 mM acetate buffer vehicle demonstrated the largest increase in solubility of Fleroxacin (from ˜0.4 mg/mL to ˜2.2 mg/mL). Based on these observations, the 50 mM acetate buffer was selected as the reconstitution vehicle for all remaining lyophilization vials. As shown in the Graph of FIG. 6, excipients like Sucrose and CAPTISOL® further improved the solubility of Fleroxacin up to ˜13-14 mg/mL.

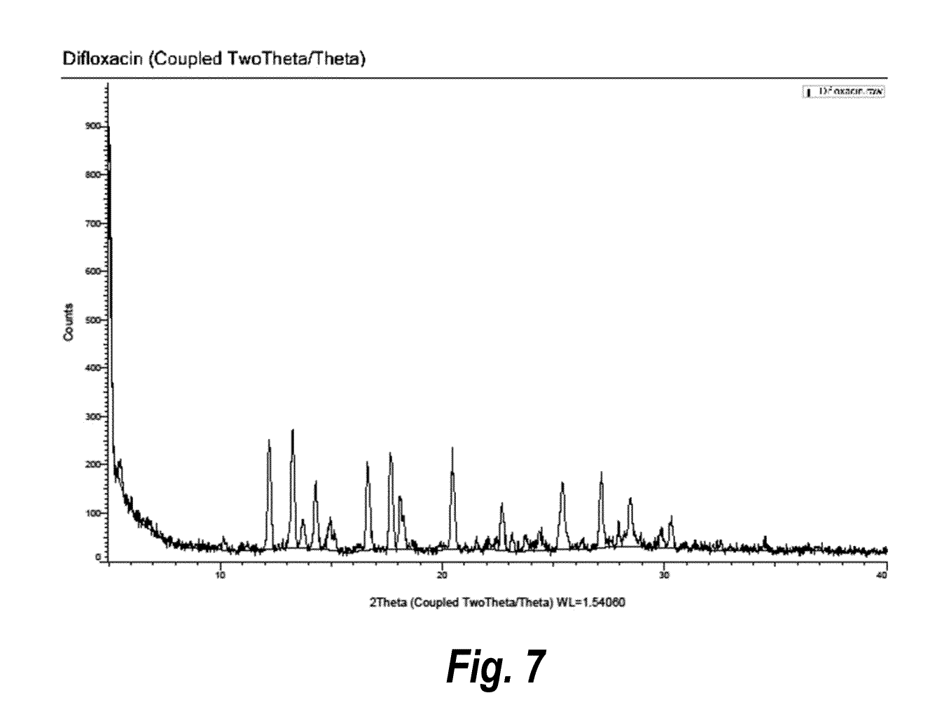

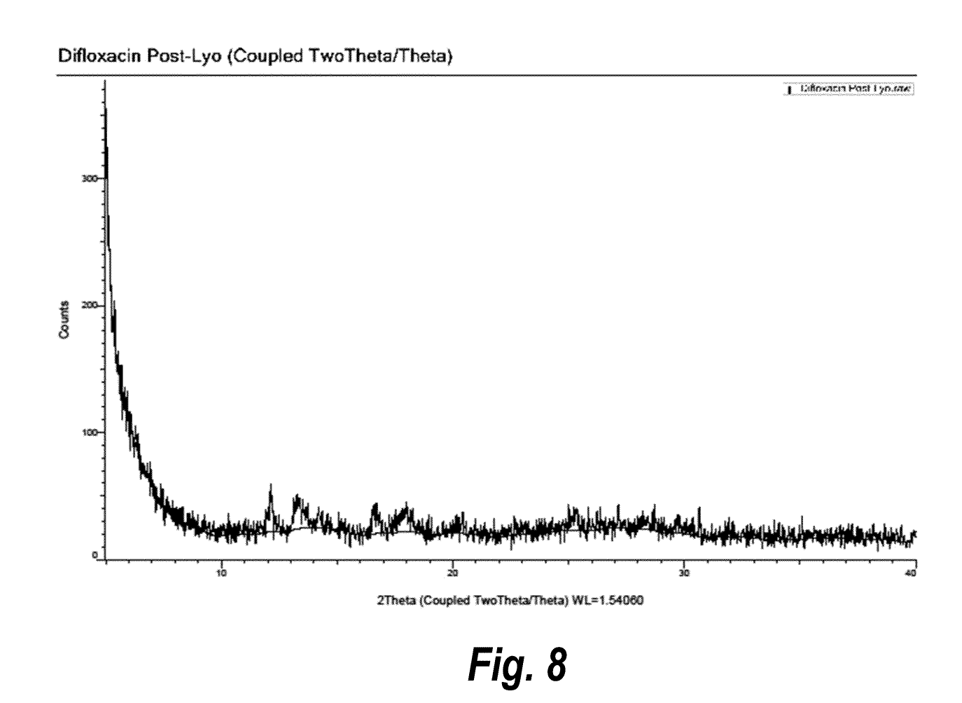

One of the main goals of this formulation screen was to improve the solubility of test compounds by converting crystalline powder forms to metastable amorphous forms. The noticeable change from a crystalline compound to an amorphous compound can be seen by a process called X-Ray Powder Diffraction (XRPD) that diffracts X-Rays on the appropriate powder sample for their structural characterization, providing graphs (see FIGS. 7 and 8). As shown in these figures, one of the test compounds (Difloxacin) was successfully converted from a crystalline form (XRPD shown in FIG. 7) to an amorphous form (XRPD shown in FIG. 8) as a result of the lyophilization process. Similar conversion to an amorphous form was observed for Paroxetine, whereas, Ciprofloxacin and Fleroxacin converted to semi-crystalline forms.

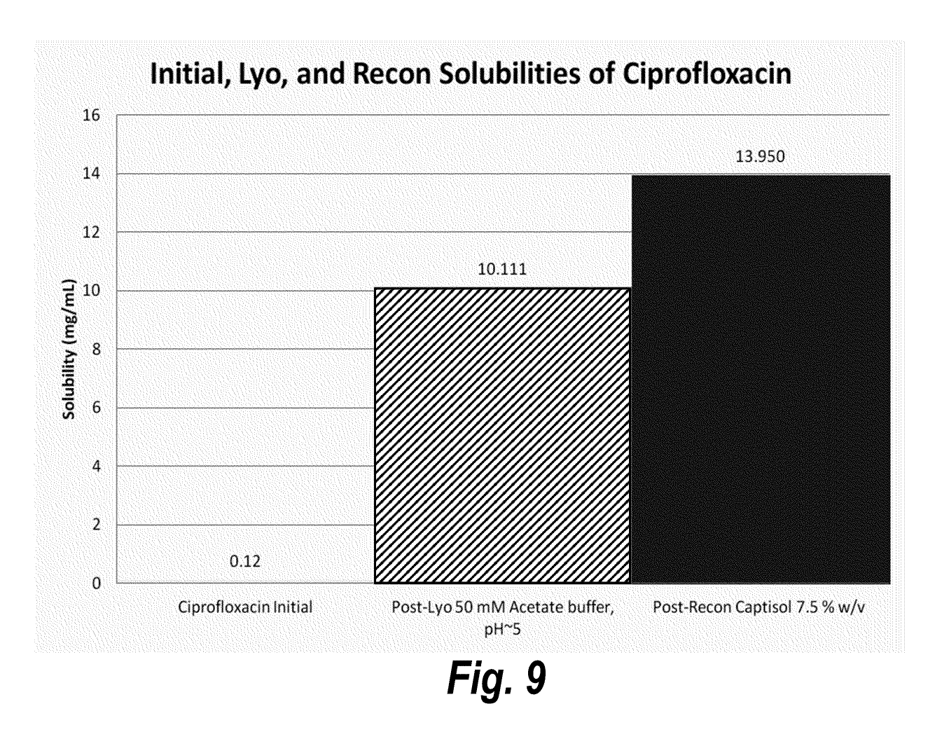

The graphs depicted in FIGS. 9, 10 and 11, demonstrated solubility improvement for the three test compounds. As shown in these plots, the HT-lyophilization approach resulted in solubility improvements of ˜100×, ˜3-4× and ˜3-4× for Ciprofloxacin, Paroxetine and Difloxacin, respectively.

FIG. 9 depicts the solubility improvement from initial solubility to post lyophilization (lyo) solubility to post-reconstitution (recon) solubility with an excipient of Ciprofloxacin

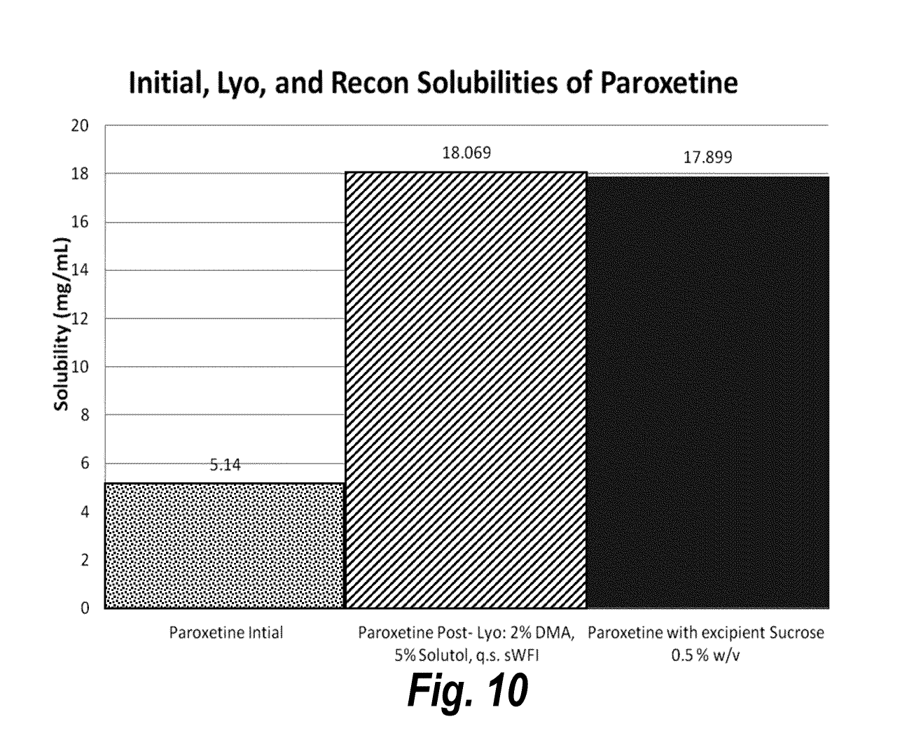

FIG. 10 depicts the solubility improvement from initial solubility to post lyophilization solubility to post-reconstitution solubility with an excipient of Paroxetine.

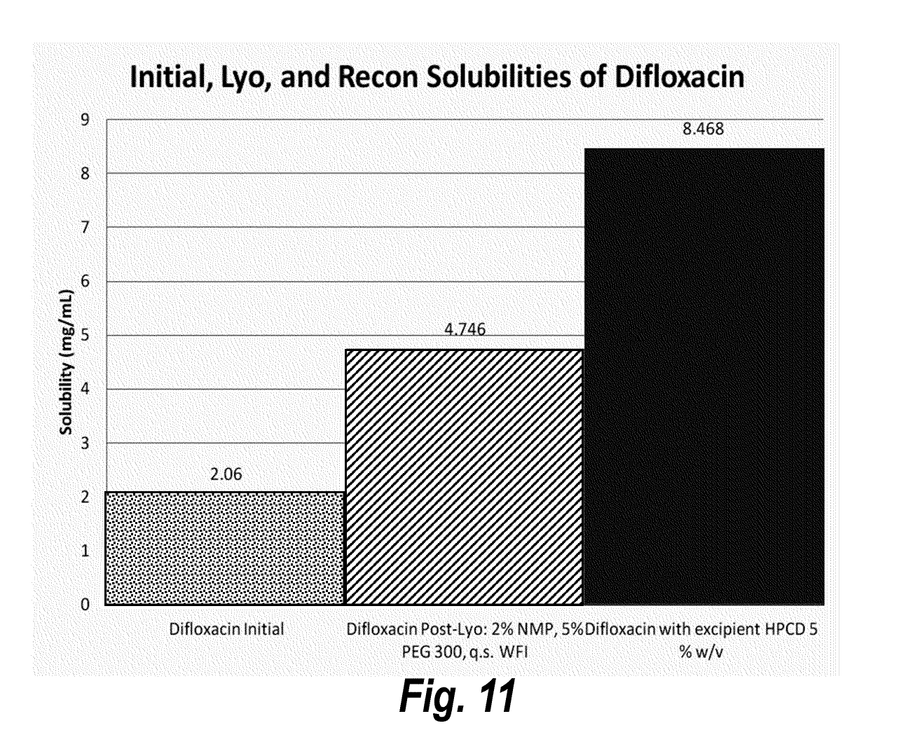

FIG. 11 depicts the solubility improvement from initial solubility to post lyophilization solubility to post-reconstitution solubility with an excipient of Difloxacin.

### Example 3

### 103-46

### Experiment Description

Aim: To screen Paroxetine and Difloxacin through the high throughput lyophilization platform to obtain a formulation at 20 mg/mL.

### Procedure: Paroxetine:

1. Weighed out 64.10 mg of the drug in a vial.
2. Added 12.82 mL of sWFI to the vial.
3. Vortexed the solution for 5 minutes.
4. A clear solution was obtained.
5. Filtered the solution through 0.22μ, filter.
6. Aliquoted 500 μL of the solution into respective vials.
7. Kept for lyophilzation.

### Difloxacin:

1. Weighed out 62.00 mg of the drug in a vial.
2. Added 12.40 mL of sWFI to the vial.
3. Vortexed the solution for 5 minutes.
4. A clear solution was obtained.
5. Filtered the solution through 0.22μ filter.
6. Aliquoted 500 μL of the solution into respective vials.
7. Kept for lyophilzation.
See the 96-well plate map below:

| | 1 | 2 | 3 | 4 | 5 | 6 | 7 | 8 | 9 | 10 | 11 | 12 |
|---|---|---|---|---|---|---|---|---|---|---|---|---|
| A | Paroxetine | | | Paroxetine | | | Paroxetine | | | Paroxetine | | |
| B | Sucrose, trehalose, | | | CAPTISOL ®, | | | Glycine, | | | | | |
| | HPBCD | | | C-17, S-630 | | | Alanine | | | | | |
| C | | | | | | | | | | | | |
| D | Difloxacin | | | Difloxacin | | | Difloxacin | | | Difloxacin | | |
| E | Sucrose, trehalose, | | | CAPTISOL ®, | | | Glycine, | | | | | |
| | HPBCD | | | C-17, S-630 | | | Alanine | | | | | |
| F | | | | | | | | | | | | |
| G | | | | | | | | | | | | |
| H | | | | | | | | | | | | |

### Example 4

### 103-53

### Experiment Description

Aim: To screen PVP C-12 as stabilizer and solubilizer instead of C-17.

### Procedure:

Weighed out 28.43 mg of the drug in a vial.
Added 10.8 mL of sWFI to the vial.
Vortexed for 5 minutes.
Sonicated for 1 minute.
Vortexed for 5 minutes.
A clear solution with few particles was observed.
Filtered the solution.
A clear solution was obtained.
Aliquotted the drug solution into different vials.
Aliquotted the excipient concentration in respective vials as shown below.
Also kept (regular lyophilization vial) one each of 2% C-17; 2% C-17+2.5% Sucrose; 2% C-12 for reconstitution tests.
   See the 96-well plate map below:

| | 1 | 2 | 3 | 4 | 5 | 6 | 7 | 8 | 9 | 10 | 11 | 12 |
|---|---|---|---|---|---|---|---|---|---|---|---|---|
| A | PVP C-17 | | | PVP C-17 | | | PVP C-17 | | | | | |
| | at 0.5% w/v | | | at 1.0% w/v | | | at 1.5% w/v | | | | | |
| B | PVP C-17 | | | PVP C-17 | | | PVP C-17 | | | PVP C-12 | | |
| | at 0.5% w/v + | | | at 1.0% w/v + | | | at 1.5% w/v + | | | at 2.0% w/v + | | |
| | 2.5% Sucrose | | | 2.5% Sucrose | | | 2.5% Sucrose | | | 2.5% Sucrose | | |
| D | PVP C-12 | | | PVP C-12 | | | PVP C-12 | | | PVP C-12 | | |
| | at 0.5% w/v | | | at 1.0% w/v | | | at 1.5% w/v | | | at 2.0% w/v | | |
| F | | | | | | | | | | | | |
| G | | | | | | | | | | | | |
| H | | | | | | | | | | | | |

Clear solutions upon reconstitution (93.5 μL) obtained in:
HT-vials: 2% C-12 PLASDONE™, 2% C-12+2.5% Sucrose, 1.5% C-17+2.5% Sucrose Regular lyophilization vials: 2% C-17 PLASDONE™, 2% C-12 PLASDONE™, 2% C-17 PLASDONE™+2.5% Sucrose

### Example 5

### 103-34

### Experiment Description

Aim: To run test compounds, cmpd 1 and cmpd 2 through the HT-Lyophilization screen.

### Procedure:

1. Weighed out 20.09 mg of cmpd 1 in a vial.
2. Added 4.560 μL of sWFi to the vial giving a 4 mg/mL solution.
3. Weighed out 18.67 mg of cmpd 2 in a vial.
4. Added 5.91 mL of sWFI to the vial to make a stock solution of 3 mg/mL.
5. Upon addition of sWFI, the vial was vortexed for 10 minutes. Most of the drug had solubilized.
6. The vial was placed in sonicator for 2 minutes.
7. The vial was vortexed for 5 minutes. A clear solution with few particles was obtained.
8. Filtered the solution using 0.22μ, filter. The solution was clear post filtration.
9. Using the below plate map as a guide, added 650 μL of the above solution into 15 vials (as shown in the 96-well plate map below). Vials in row A, columns 1-5 and in row B, columns 1-10 were vials with the drug.
10. Added respective amount of excipient concentrates into the vials (using the SOP as a reference).
11. According to calculations, after the completion of the lyophilization cycle each vial in which drug was added would have 2.6 mg of '172 and 1.95 mg of '099 drug material.
12. Set up the lyophilization cycle using standard operating parameters set forth in Example 1 as a reference on the AdVantage™ Plus Bench top lyo.

| | 1 | 2 | 3 | 4 | 5 | 6 | 7 | 8 | 9 | 10 | 11 | 12 |
|---|---|---|---|---|---|---|---|---|---|---|---|---|
| A | ‘172 + | Drug in | Drug in | Drug in | Drug in | sWFI | sWFI | sWFI | sWFI | sWFI | sWFI | sWFI |
| | PVP | sWFI | sWFI | sWFI | sWFI | | | | | | | |
| B | 1% | 1% | 1% | 1.5% | 1.5% | 0.4% | 0.4% | 0.4% | 80 mM | 80 mM | sWFI | sWFI |
| | Sucrose | Trehalose | Mannitol | HPBCD | CAPTISOL ® | PVP | PLASDONE ™ | Poloxamer | Glycine | Alanine | | |
| | | | | | | | S-630 | | | | | |

| | | | | |
|---|---|---|---|---|
| C | Sucrose 2% w/v | Sucrose 2% w/v | Trehalose 2% w/v | Trehalose 2% w/v |
| D | HPCD 1.5% w/v | HPCD 1.5% w/v | CAPTISOL ® 1.5% w/v | CAPTISOL ® 1.5% |
| | | | | w/v |
| E | PLASDONE ™ S-630 at | PLASDONE ™ S-630 at | 2% Mannitol | 2% Mannitol |
| | 0.4% w/v | 0.4% w/v | | |
| F | PVP C-17 at 0.4% w/v | PVP C-17 at 0.4% w/v | Poloxamer 0.4% w/v | Poloxamer 0.4% w/v |
| G | 80 mM Glycine | 80 mM Glycine | 80 mM Alanine | 80 mM Alanine |
| H | | | | |

### Reconstitution Trials #1 ('172):

### 1) DF #1 (PVP)

a. Added 130 μl of sWFI to the vial and vortexed for 5 minutes.
b. A slightly hazy solution was obtained.
c. Measured the pH of the solution and found it to be ˜5.05
d. Filtered the solution and submitted the solution for analysis.

### 2) DF #2 (Sucrose)

a. Added 117 μL of 2% NMP, 5% PEG 300, q.s. sWFI to the vial and vortexed for 2 minutes. A clear solution was obtained within 5 seconds.
b. A clear solution was obtained.
c. Measured the pH of the solution and found it to be 5.2.
d. Filtered the solution and submitted the solution to for analysis.

### 3) DF #3 (PVP)

a. Added 117 μL of 2% NMP, 5% PEG 300, q.s. sWFI to the vial and vortexed for 2 minutes.
b. A clear solution was obtained.
c. Measured the pH of the solution and found it to be 5.2.
d. Filtered the solution and submitted the solution to for analysis.

### 4) DF #4 (Sucrose_with ˜2 mg)

a. Added 90 μl of sWFI to the vial and vortexed for 5 minutes.
b. A clear solution was obtained within 5 seconds.
c. Measured the pH of the solution and found it to be 5.2.
d. Filtered the solution and submitted the solution to for analysis.

### Reconstitution Trials #1 ('099):

### 1) DF #1 (PVP)

a. Added 97.5 μl of 2% NMP, 5% PEG 300, q.s. sWFI to the vial and vortexed for 5 minutes.
b. A clear solution was obtained.
c. Measured the pH of the solution and found it to be ˜5.05
d. Filtered the solution and submitted the solution to for analysis.

### 2) DF #2 (PLASDONE™)

a. Added 97.54 μL of 2% NMP, 5% PEG 300, q.s. sWFI to the vial and vortexed for 2 minutes. A clear solution was obtained within 5 seconds.
b. A clear solution was obtained.
c. Measured the pH of the solution and found it to be 5.2.
d. Filtered the solution and submitted the solution for analysis.

### 3) DF #3 (Glycine)

a. Added 97.5 μL of 2% NMP, 5% PEG 300, q.s. sWFI to the vial and vortexed for 2 minutes.
b. A hazy solution was obtained.
c. Measured the pH of the solution and found it to be 5.2.
d. Filtered the solution.

### Example 6

### 103-29

### Experiment Description

Aim: To run test compound, cmpd 3, through the HT-Lyophilization screen.

### Procedure:

1. Weighed out 39.85 mg of cmpd 3 in a vial.
2. Added 12.43 mL of sWFI to the vial to make a stock solution of 3 mg/mL (WBP: 936 μg/mg).
3. Upon addition of sWFI, the vial was vortexed for 10 minutes. Most of the drug had solubilized.
4. The vial was placed in a sonicator for 2 minutes.
5. The vial was vortexed for 5 minutes. A clear solution with few particles was obtained.
6. Filtered the solution using 0.22μ, filter. The solution was clear post filtration.
7. Using the below plate map as a guide, added 650 μL of the above solution into 17 vials (as shown in the 96-well plate map below). Vials in row A, columns 1-7 and in row B, columns 1-10 were vials with the drug.
8. Added respective amount of excipient concentrates into the vials (using the SOP as a reference).
9. According to calculations, after the completion of the lyophilization cycle each vial in which drug was added would have 1.95 mg of drug material.
10. Set up the lyophilization cycle using Example 1 cycles as a reference.
See 96-well plate map below:

| | 1 | 2 | 3 | 4 | 5 | 6 | 7 | 8 | 9 | 10 | 11 | 12 |
|---|---|---|---|---|---|---|---|---|---|---|---|---|
| A | Drug in | Drug in | Drug in | Drug in | Drug in | Drug in | Drug in | sWFI | sWFI | sWFI | sWFI | sWFI |
| | sWFI | sWFI | sWFI | sWFI | sWFI | sWFI | sWFI | | | | | |
| B | 1% | 1% | 1% | 1.5% | 1.5% | 0.4% | 0.4% | 0.4% | 80 mM | 80 mM | sWFI | sWFI |
| | Sucrose | Trehalose | Mannitol | HPBCD | CAPTISOL ® | PVP | PLASDONE ™ | Poloxamer | Glycine | Alanine | | |
| | | | | | | | S-630 | | | | | |

| | | | | |
|---|---|---|---|---|
| C | Sucrose 2% w/v | Sucrose 2% w/v | Trehalose 2% w/v | Trehalose 2% w/v |
| D | HPCD 1.5% w/v | HPCD 1.5% w/v | CAPTISOL ® 1.5% w/v | CAPTISOL ® 1.5% |
| | | | | w/v |
| E | PLASDONE ™ S-630 | PLASDONE ™ S-630 at 0.4% | 2% Mannitol | 2% Mannitol |
| | at 0.4% w/v | w/v | | |
| F | PVP C-17 at 0.4% w/v | PVP C-17 at 0.4% w/v | Poloxamer 0.4% w/v | Poloxamer 0.4% w/v |
| G | | | | |
| H | | | | |

1. Upon completion of lyophilization cycle, the vials with drug in sWFI didn't have uniform cakes.
2. The ones with excipient had nice uniform cakes.

### Reconstitution Trial #1;

1. After the lyophilization cycle as per calculations, each vial with the drug would have 1.95 mg of solid active pharmaceutical ingredient (API).

2. Initially the “Drug in sWFI” vials were tried with following 5 vehicles targeting 20 mg/mL.

a. sWFI:
b. 50 mM Acetate buffer

### c. 10% CREMOPHOR®

d. 3% NMP, 5% PEG300, q.s. sWFI
e. 3% DMA, 5% SOLUTOL®, q.s sWFI

3. None of the samples gave clear solutions. All of them were milky/hazy. The ones with DMA and NMP looked less hazy.

4. All of the samples were filtered using the centrifuge filters.

5. All of the samples were analyzed using HPLC.

### Results #1:

| | Conc. |
|---|---|
| Vehicle | (mg/mL |
| sWFI | 4.666 |
| 50 mM Acetate buffer, pH 5 | 5.542 |
| 10% CREMOPHOR ® in sWFI | 4.735 |
| 2% DMA, 5% SOLUTOL ®, sWFI | 5.583 |
| 2% NMP, 5% PEG 300, sWFI | 5.839 |

### Trial #2

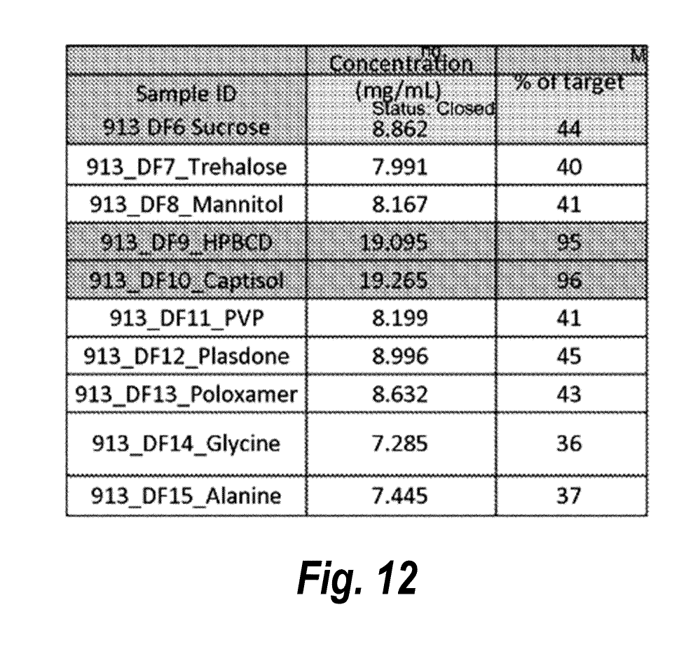

1. Since Vehicle number 5 (2% NMP, 5% PEG 300, sWFI) gave the maximum concentration for the first set of vials, the rest of the vials were reconstituted using that vehicle.
2. A stock solution of vehicle was made.
3. After adding 97.5 μl of vehicle to the vials, the following observations were made:
a. Sucrose: hazy
b. Trehalose: hazy
c. Mannitol: hazy
d. HPBCD: clear
e. CAPTISOL®: clear
f. PVP: hazy
g. PLASDONE™: hazy
h. Poloxamer: hazy
i. Glycine: hazy
j. Alanine: hazy
4. For all of the formulations a common observation was that the drug was solubilized in all of the vials above but crashed or precipitated out of the solution within 30 seconds. These results are summarized in the table depicted in FIG. 12.

### Results #2

The maximum concentration as seen was obtained with 7.5% HPBCD and 7.5% CAPTISOL®.

### Example 7

### 103-43

### Experiment Description

Aim: To prepare a screen of a test compound, cmpd 2, using the HT-Lyophilization screen.

### Procedure:

Weighed out 26.32 mg of material.

Added 10 mL of sWFI to the vial and vortexed for 5 minutes.

Sonicated for 1 minute.

Vortexed for 5 minutes.

A clear solution with few crystals was obtained.

Filtered the solution.

Aliquoted 750 μl into the HT-Lyophilization vials.

The 96-well plate map is shown below:

| | 1 | 2 | 3 | 4 | 5 | 6 | 7 | 8 | 9 | 10 | 11 | 12 |
|---|---|---|---|---|---|---|---|---|---|---|---|---|
| A | PVP C-17 | | | PVP C-17 | | | | | | | | |
| | at 2% w/v | | | at 2% w/v | | | | | | | | |
| B | | | | | | | PLASDONE ™ | | | | | |
| | | | | | | | S 630 2% | | | | | |
| C | | | | | | | | | | | | |
| D | | | | | | | | | | | | |
| E | | | | | | | | | | | | |
| F | | | | | | | | | | | | |
| G | | | | | | | | | | | | |
| H | | | | | | | | | | | | |

### Reconstitution Trials: PVP:

1. 0.5% NMP, 5% PEG 300 and sWFI.

The vial was supposed to have 1.875 mg of material.

Added 93.5 μl of vehicle.

A slightly hazy solution was obtained.

2. 1% NMP, 5% PEG 300 and sWFI.

The vial was supposed to have 1.875 mg of material.

Added 93.5 μl of vehicle.

A slightly hazy solution was obtained.

3. 2% NMP, 5% PEG 300 and sWFI.

The vial was supposed to have 1.875 mg of material.

Added 93.5 μl of vehicle.

A clear solution was obtained.

Filtered and analyzed with HPLC.
PLASDONE™ vials:

1. 0.5% NMP, 5% PEG 300 and sWFI.

a. The vial was supposed to have 1.875 mg of material.
b. Added 93.5 μl of vehicle.
c. A slightly hazy solution was obtained.

2. 1% NMP, 5% PEG 300 and sWFI.

a. Added 93.5 μL of vehicle.
b. A clear solution was obtained.
c. Filtered and analyzed using HPLC.

### Results:

| | Conc. (mg/mL) | | | |
|---|---|---|---|---|
| Excipient | T0_1 | T0_2 | T0_3 | Mean |
| PLASDONE ™ | 19.42 | 19.48 | 19.46 | 19.45 |
| C-17 | | | | |
| PLASDONE ™ | 19.11 | 19.1 | 19.1 | 19.10 |
| S630 | | | | |

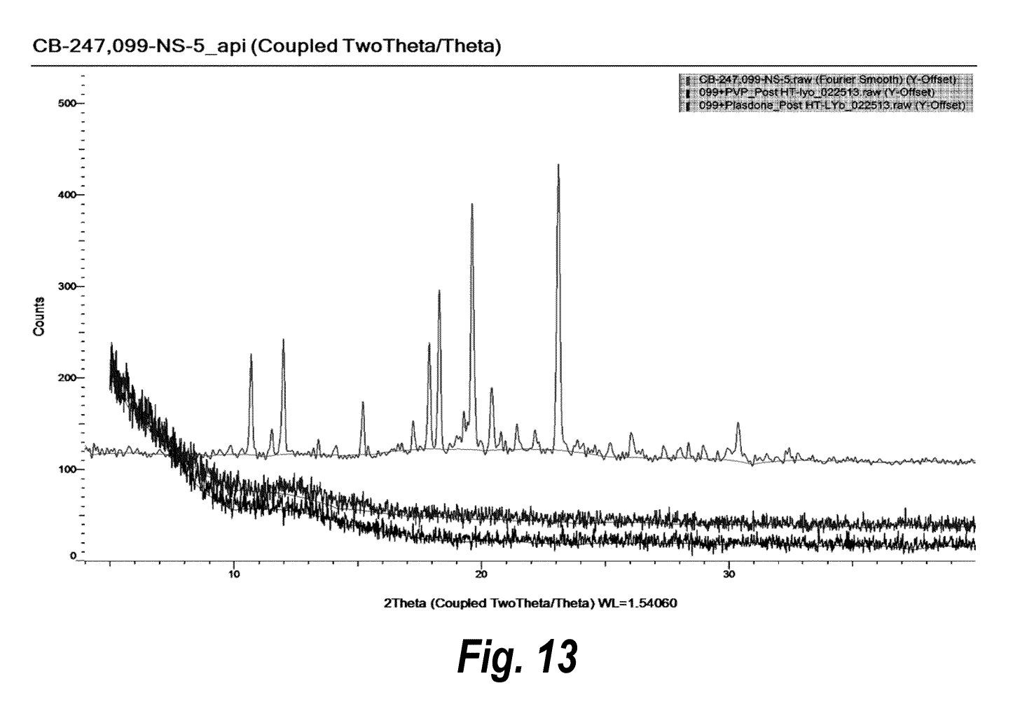

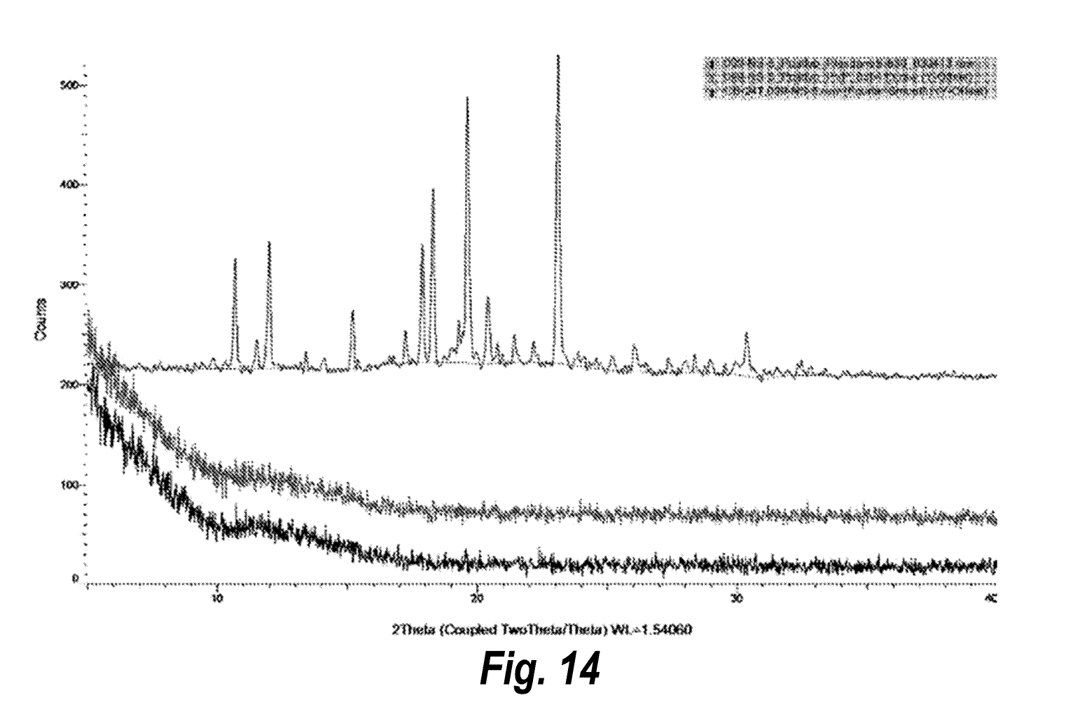

The samples precipitated at T=2 hours. The samples were stored at 4 C., which might have been the cause of precipitation. A follow up formulation would be analyzed by keeping it at room temperature. XRPD results are depicted in FIG. 13 and FIG. 14.

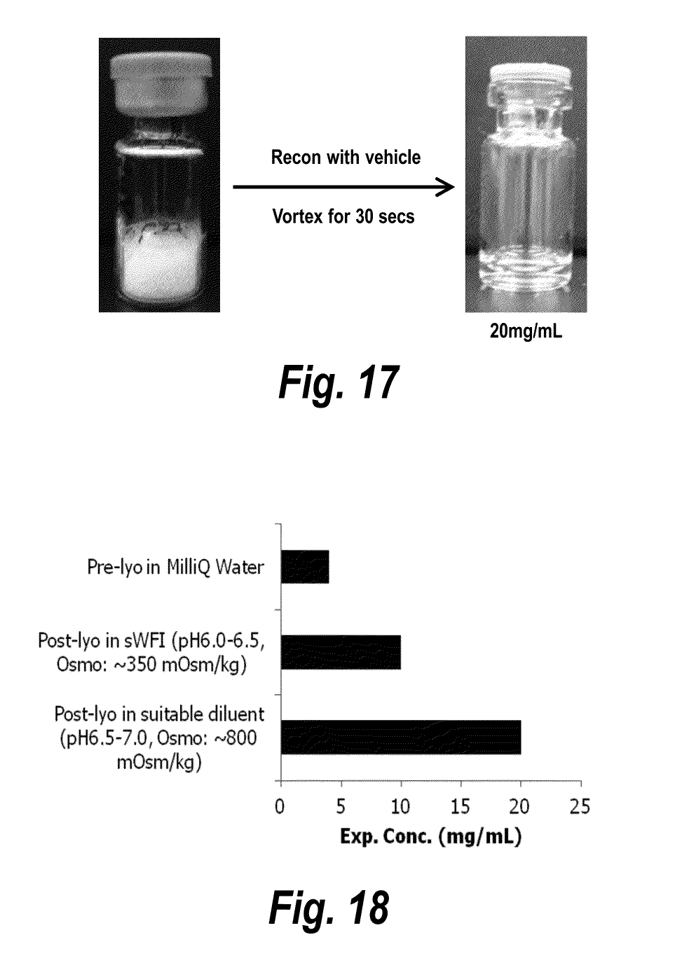

FIG. 17 depicts reconstitution of a select compound of interest in a formulation where the vial was reconstituted with a vehicle after the vial was vortexed for about 30 seconds. The resulting solution was clear. The solubility of cmpd 2 improved post lyophilization in sWFI with greater improvement post lyophilization in a diluent compared to pre-lyophilization in MilliQ water (FIG. 18).

### Batch #2

Aim: To prepare screen of a test compound, cmpd 2, through the HT-Lyophilization screen.

### Procedure:

Weighed out 33.20 mg of material.

Added 12.61 mL of sWFI to the vial and vortexed for 5 minutes.

Sonicated for 1 minute.

Vortexed for 5 minutes.

A clear solution with few crystals was obtained.

Filtered the solution.

Aliquoted required amount of excipient concentration into the vials.

Aliquoted 750 μl into the respective HT-Lyophilization vials, and vortexed the solutions.

Lyophilized using the recipe 2 in the Bench-top lyophilizer.

The 96-well plate map is shown below:

| | 1 | 2 | 3 | 4 | 5 | 6 | 7 | 8 | 9 | 10 | 11 | 12 |
|---|---|---|---|---|---|---|---|---|---|---|---|---|
| A | PVP C-17 at 2% w/v | | | PVP C-17 at | | | | | | | | |
| B | Sucrose 5% | | | 2% w/v | | | | | | | | |
| C | PVP C-17 at 2% w/v + | | | | | | | | | | | |
| | Sucrose 2.7% | | | | | | | | | | | |
| D | PLASDONE ™ S 630 | | | PLASDONE ™ | | | | | | | | |
| E | Sucrose 2.7% + | | | S 630 | | | | | | | | |
| | PLASDONE ™ S 630 | | | | | | | | | | | |
| F | | | | | | | | | | | | |
| G | | | | | | | | | | | | |
| H | | | | | | | | | | | | |

### Reconstitution Trials: 2% PVP:

4. 1% DMA, 10% PEG 300 and sWFI.

The vial was supposed to have 1.875 mg of material.

Added 93.5 μl of vehicle.

A slightly hazy solution was obtained.

Added 93.75 μl of vehicle.

A clear solution was obtained.

5. 2% DMA, 10% PEG 300 and sWFI.

The vial was supposed to have 1.875 mg of material.

Added 93.5 μl of vehicle.

A clear solution was obtained.

6. 1% NMP, 10% PEG 300 and sWFI

Added 93.5 μL of vehicle.

A clear solution was formed.

Filtered the solution and measured the pH and osmolality.

Osmolality found to be 624 mOsm/kg.

### 2% PVP+2.7% Sucrose:

1. 2% DMA, 10% PEG 300 and sWFI.

The vial was supposed to have 1.875 mg of material.

Added 93.5 μl of vehicle.

A clear solution was obtained.

### 5% Sucrose:

1. 2% DMA, 10% PEG 300 and sWFI.

The vial was supposed to have 1.875 mg of material.

Added 93.5 μl of vehicle.

A hazy solution was obtained.

Not submitted.

### 2% PLASDONE™:

1. 2% DMA, 10% PEG 300 and sWFI.

The vial was supposed to have 1.875 mg of material.

Added 93.5 μl of vehicle.

A clear solution was obtained. 2% PLASDONE™+2.7% Sucrose:

2. 2% DMA, 10% PEG 300 and sWFI.

The vial was supposed to have 1.875 mg of material.

Added 93.5 μl of vehicle.

A hazy solution was obtained.

Not submitted.

### Example 8

### 103-28

Aim: To run a test compound, cmpd 2, through the HT-Lyophilization screen.

### Procedure:

1. Weighed out 35.31 mg of cmpd 2, 099-NS-5 in a vial.
2. Added 11.18 mL of sWFI to the vial to make a stock solution of 3 mg/mL.
3. Upon the addition of sWFI, the vial was vortexed for 10 minutes. Most of the drug had solubilized.
4. The vial was placed in a sonicator for 2 minutes.
5. The vial was vortexed for 5 minutes. A clear solution with few particles was obtained.
6. Filtered the solution using 0.22μ, filter. The solution was clear post filtration.
7. Using the below plate map as a guide, added 650 μL of the above solution into 15 vials (as shown in the 96-well plate map below). Vials in row A, columns 1-5 and in row B, columns 1-10 were vials with the drug.
8. Added respective amount of excipient concentrates into the vials (Using the SOP as a reference).
9. According to calculations, after the completion of the lyophilization cycle each vial in which drug was added would have 1.95 mg of drug material.
10. Set up the lyophilization cycle using the Example 1 cycle as a reference.

| | 1 | 2 | 3 | 4 | 5 | 6 | 7 | 8 | 9 | 10 | 11 | 12 |
|---|---|---|---|---|---|---|---|---|---|---|---|---|
| A | Drug in | Drug in | Drug in | Drug in | Drug in | sWFI | sWFI | sWFI | sWFI | sWFI | sWFI | sWFI |
| | sWFI | sWFI | sWFI | sWFI | sWFI | | | | | | | |
| B | 1% | 1% | 1% | 1.5% | 1.5% | 0.4% | 0.4% | 0.4% | 80 mM | 80 mM | sWFI | sWFI |
| | Sucrose | Trehalose | Mannitol | HPBCD | CAPTISOL ® | PVP | PLASDONE ™ | Poloxamer | Glycine | Alanine | | |
| | | | | | | | S-630 | | | | | |

| | | | | |
|---|---|---|---|---|
| C | Sucrose 2% w/v | Sucrose 2% w/v | Trehalose 2% w/v | Trehalose 2% w/v |
| D | HPCD 1.5% w/v | HPCD 1.5% w/v | CAPTISOL ® 1.5% w/v | CAPTISOL ® 1.5% |
| | | | | w/v |
| E | PLASDONE ™ S-630 | PLASDONE ™ S-630 | 2% Mannitol | 2% Mannitol |
| | at 0.4% w/v | at 0.4% w/v | | |
| F | PVP C-17 at 0.4% w/v | PVP C-17 at 0.4% w/v | Poloxamer 0.4% w/v | Poloxamer 0.4% w/v |
| G | 80 mM Glycine | 80 mM Glycine | 80 mM Alanine | 80 mM Alanine |
| H | | | | |

Upon completion of lyophilization cycle, the vials with drug in sWFI didn't have uniform cakes.
1. The ones with excipient had nice uniform cakes.

Reconstitution Trials:

### HPLC set up:

1. Weighed out 1.31 mg as standard.
2. Added 1 mL of MilliQ water to it.
3. Standards were: STD_L: 0.06 mg/mL, STD_M: 0.65 mg/mL, STD_H: 1.31 mg/mL.
4. The samples were diluted 50× before putting them into HPLC.

### Samples:

1. After the lyophilization cycle as per calculations, each vial with the drug would have 1.95 mg of solid API.

2. Initially the “Drug in sWFI” vials were tried with following 5 vehicles targeting 20 mg/mL

a. sWFI
b. 50 mM Acetate buffer

### c. 10% CREMOPHOR®

d. 3% NMP, 5% PEG 300, q.s. sWFI
e. 3% DMA, 5% SOLUTOL®, q.s sWFI

3. None of the samples gave clear solutions. The one with DMA and NMP looked the most clear.

4. All of the samples were filtered using the centrifuge filters.

5. All of the samples were analyzed by HPLC.

### Results #1:

| | Conc. |
|---|---|
| Vehicle | (mg/mL |
| sWFI | 11.4 |
| 50 mM Acetate buffer, pH 5 | 12.72 |
| 10% CREMOPHOR ® in sWFI | 12.41 |
| 2% DMA, 5% SOLUTOL ®, sWFI | 14.3 |
| 2% NMP, 5% PEG 300, sWFI | 14.87 |

### Trial #2

1. Since Vehicle number 5 (2% NMP, 5% PEG 300, sWFI) gave the maximum concentration for the first set of vials, the rest of the vials were reconstituted using that vehicle.
2. A stock solution of vehicle was made.
3. After adding 97.5 μl of vehicle to the vials, the following observations were made:
a. Sucrose: milky
b. Trehalose: milky
c. Mannitol: milky
d. HPBCD: clear
e. CAPTISOL®: clear
f. PVP: clear
g. PLASDONE™: hazy
h. Poloxamer: milky
i. Glycine: milky
j. Alanine: milky

### Results #2:

| | Vial #1 | Vial #2 | Mean | |
|---|---|---|---|---|
| Excipient | (mg/mL) | (mg/mL) | (mg/mL) | Observation |
| Sucrose | 13.1 | 13.0 | 13.0 | milky |
| Trehalose | 13.4 | 13.4 | 13.4 | milky |
| Mannitol | 13.2 | 13.1 | 13.2 | milky |
| HPBCD | 18.5 | 18.4 | 18.5* | clear |
| CAPTISOL ® | 18.8 | 18.8 | 18.8* | clear |
| PVP C-17 * | 19.0 | 18.9 | 19.0* | clear* |
| PLASDONE ™ | 18.5 | 18.4 | 18.5* | hazy |
| Poloxamer | 15.0 | 14.8 | 14.9 | milky |
| Glycine | 16.4 | 16.3 | 16.4 | milky |
| Alanine | 16.2 | 16.2 | 16.2 | milky |

Samples with the highest mean concentration and clarity are marked with asterisks.

### Example 9

### 103-51

Aim: To generate stability data on a test compound, cmpd 2, for the lyophilized material.

### Procedure:

1. Weighed out 31 mg of the material in a vial.
2. Added 11.78 mL of sWFI to the vial.
3. Vortexed the vial for 5 minutes.
4. Sonicated the vial for 1 minute.
5. Vortexed for 5 minutes.
6. Filtered the solution through 0.22μ, filter.
7. A clear solution was obtained.

### Example 10

### 103-22 run 5

Aim: To set up a lyophilization cycle with actual drug substance and evaluate the reconstitution behavior of the material obtained.
Procedure (pre-lyophilization):
1. Weighed out 38.64 mg of the drug in a vial.
2. Prepared a stock solution of CB-243,172-NS-12 in sWFI at 5 mg/mL.
3. Filtered the solution using 0.22μ, filter. The solution was clear pre and post filtration.
4. Using the below plate map as a guide, added 500 μL of the above solution into 18 vials (as shown in the 96-well plate map below). Vials in row A, columns 1-5 and in row B, columns 1-9 were vials with the drug.
5. Added respective amount of excipient concentrates into the 18 vials.
6. Filled up the rest of the vials as blanks with excipient solutions.
7. Set up the lyophilization cycle using Example 1 cycle as a reference.

| | 1 | 2 | 3 | 4 | 5 | 6 | 7 | 8 | 9 | 10 | 11 | 12 |
|---|---|---|---|---|---|---|---|---|---|---|---|---|
| A | Drug in | Drug in | Drug in | Drug in | Drug in | sWFI | sWFI | sWFI | sWFI | sWFI | sWFI | sWFI |
| | sWFI | sWFI | sWFI | sWFI | sWFI | | | | | | | |
| B | 2% | 2% | 1.5% | 1.5% | 0.4% | 0.4% | 80 mM | 80 mM | sWFI | sWFI | sWFI | sWFI |
| | Sucrose | Trehalose | HPBCD | CAPTISOL ® | PVP | PLASDONE ™ | Glycine | Alanine | | | | |
| | | | | | | S-630 | | | | | | |

| | | | | |
|---|---|---|---|---|
| C | Sucrose 2% w/v | Sucrose 2% w/v | Trehalose 2% w/v | Trehalose 2% w/v |
| D | HPCD 1.5% w/v | HPCD 1.5% w/v | CAPTISOL ® 1.5% | CAPTISOL ® 1.5% |
| | | | w/v | w/v |
| E | PVP C-17 at 0.4% w/v | PVP C-17 at 0.4% w/v | sWFI | sWFI |
| F | PLASDONE ™ S-630 | PLASDONE ™ S-630 | Poloxamer 0.4% w/v | Poloxamer 0.4% |
| | at 0.4% w/v | at 0.4% w/v | | w/v |
| G | 80 mM Glycine | 80 mM Glycine | 80 mM Alanine | 80 mM Alanine |
| H | | | | |

### Observation:

All of the vials were nicely dried.

Uniform cakes were formed.

In some of the vials, the cakes were cracked from the center.

### Reconstitution Trails (I):

1. 1.25 mg of test cmpd 1 was provided as a STD.

2. After the lyophilization cycle as per calculations, each vial with the drug would have 2.5 mg of solid API.

3. Initially the “Drug in sWFI” vials were tried with following 5 vehicles targeting 20 mg/mL (WBP used: 902 ug/ug).

a. sWFI
b. 50 mM Acetate buffer

### c. 10% CREMOPHOR®

d. 3% NMP, 5% PEG 300, q.s. sWFI
e. 3% DMA, 5% SOLUTOL®, q.s sWFI

4. The one which gave clear solution and 100% recovery when analyzed using HPLC, was tried for the other vials containing drug and excipients.

5. Which was 10% CREMOPHOR® in sWFI.

6. The other 9 vials were tried with 10% CREMOPHOR® in sWFI solution.

7. Filtered the solution and submitted the solution for T0 concentration analysis.

### HPLC Results:

| Sample | Conc. (mg/mL) |
|---|---|
| 172 DF sWFI | 24.8 |
| 172 DF Acetate | 24.6 |
| 172 DF DMA/SOLUTOL ® | 17.4 |
| 172 DF NMP/PEG | 24.6 |
| 172 DF 10% CREMOPHOR ® | 26.2 |

Here visually the 10% CREMOPHOR® sample was the most clear solution.

Upon adding DMA and NMP to the amorphous material, a yellow colored solution was formed, which could be the drug being degraded.

### Reconstitution Trials (II):

These 9 vials were also assumed to have 2.5 mg of API in them along with respective excipients. All the vials were reconstituted using 10% CREMOPHOR® in sWFI targeting 20 mg/mL. The WBP used for these vials was 902 μg/μg.

Observations:

1. Sucrose: clear
2. Trehalose: clear
3. HPBCD: clear

### 4. CAPTISOL®

5. PVP: clear
6. PLASDONE™ S-630: hazy
7. Poloxamer: hazy
8. Glycine: hazy
9. Alanine: clear

### HPLC Results:

| Sample | Conc. (mg/mL) |
|---|---|
| 172 DF Poloxamer | 22.6 |
| 172 DF Alanine | 22.7 |
| 172 DF CAPTISOL ® | 23.4 |
| 172 DF | 22.4 |
| 172 DF Sucrose | 22.9 |
| 172 DF Glycine | 23.2 |
| 172 DF Trehalose | 23.2 |
| 172 DF HP-B-CD | 23.4 |
| 172 DF PVP | 23.5 |

NOTE: The higher concentration obtained as compared to the target concentration can be attributed to the higher WBP of the post lyophilized material, which was not measured and accounted.
An example 96-well plate map is provided below:

| | 1 | 2 | 3 | 4 | 5 | 6 | 7 | 8 | 9 | 10 | 11 | 12 |
|---|---|---|---|---|---|---|---|---|---|---|---|---|
| A | sWFI | sWFI | sWFI | sWFI | sWFI | sWFI | sWFI | sWFI | sWFI | sWFI | sWFI | sWFI |

| | | | | |
|---|---|---|---|---|
| B | Sucrose 5% w/v | Sucrose 10% w/v | Sucrose 15% w/v | Sucrose 20% w/v |
| C | Trehalose 5% w/v | Trehalose 10% w/v | Trehalose 15% w/v | Trehalose 20% w/v |
| D | PVP K 15 at 0.5% w/v | PVP K 15 at 1.0% w/v | PVP K 15 at 1.5% w/v | PVP K 15 at 2.0% w/v |
| E | HPCD 1% w/v | HPCD 2.5% w/v | HPCD 5% w/v | HPCD 7.5% w/v |
| F | CAPTISOL ® 1% w/v | CAPTISOL ® 2.5% w/v | CAPTISOL ® 5% w/v | CAPTISOL ® 7.5% w/v |
| G | PLASDONE ™ S-630 | PLASDONE ™ S-630 | PLASDONE ™ S-630 | PLASDONE ™ S-630 |
| | at 0.5% w/v | at 1.0% w/v | at 1.5% w/v | at 2.0% w/v |
| H | Poloxamer 188 at | Poloxamer 188 at | Poloxamer 188 at | Poloxamer 188 at |
| | 0.5% w/v | 1% w/v | 1.5% w/v | 2% w/v |

Thus we have described in detail features and advantages of the present method of making formulations for compounds of interest with the understanding that the methods are capable of being modified and altered without departing from the teaching herein. Therefore the present invention should not be limited to the detail description herein but should encompass the subject matter of the claims that follow and their equivalents.

1. A method for identifying one or more solutions for rehydration, excipients and diluents for use with a compound of interest in a lyophilization process and rehydration process, for parenteral formulations with such compound of interest, said method comprising the steps of:
a. providing a plurality of wells for performing lyophilization processes, said plurality of wells divided into well groups, said well groups corresponding to at least a first well group and at least one second well group, said first well group having at least one well for receiving each test rehydrating solution, said second well group having at least one well for receiving a quantity of a test excipient and/or diluent;
b. placing an aliquot of a compound of interest to each well of said first well group and said second well group, said aliquot having a concentration of the compound of interest at or above the desired concentration of a parenteral formulation, and placing a quantity of a test excipient and/or diluent to each wells of said second well group, and placing the contents of all wells of the first well group and the second well group in solution;
c. imposing lyophilization conditions on said plurality of wells to produce a plurality of lyophilized samples comprising lyophilized compound of interest in each well of said first well group, and lyophilized compound of interest with a test excipient and/or diluent in each well of said second well group;
d. rehydrating a well of said first well group with each test rehydrating solution and identifying at least one preferred rehydrating solution; and
e. rehydrating each well of said second group of wells with said at least one preferred rehydrating solution to identify at least one preferred rehydrating solution and at least one preferred excipient and/or diluent for use with a parenteral formulation of the compound of interest.

2. The method of claim 1 wherein said preferred rehydrating solution is chosen by at least one criteria from the group consisting of stability in solution, clarity and concentration of the compound of interest.

3. The method of claim 1 wherein said plurality of wells are constructed and arranged in a 96-well format.

4. The method of claim 1 wherein said lyophilization conditions are imposed by an automated process.

5. The method of claim 1 wherein said preferred excipient and/diluent and preferred rehydrating solution is used to optimize lyophilization cycle parameters.

6. The method of claim 1 wherein said step of rehydrating is performed after a period of time representing the desired stability of the lyophilized sample.

7. The method of claim 1 wherein said rehydrated sample is ranked by stability.

8. A compound of interest in a formulation having excipients and/or diluents which is lyophilized and reconstituted with a rehydrating solution before use, in which the excipients/diluents are identified by the process of claim 1.

9. A lyophilized compound of interest in which the rehydrating solution is identified by the process of claim 1.

10. A method of increasing the solubility of a pharmaceutical agent, comprising:
a. dissolving a pharmaceutical agent in a first solute, the pharmaceutical agent having a first solubility in a first solute; and
b. lyophilizing the pharmaceutical agent in the first solute to form a lyophilized composition comprising the pharmaceutical agent, the lyophilized composition having a second solubility in the first solute that is greater than the first solubility.

11. The method of claim 10, wherein the first solute is water and the lyophilized composition is obtained by lyophilizing an aqueous solution of the pharmaceutical agent.

12. The method of claim 10, wherein the pharmaceutical agent is selected from the group consisting of ciprofloxacin, paroxetine, and difloxacin.

13. The method of claim 10, wherein the method further comprises adding one or more excipients to the first solute prior to lyophilization.
